(19) United States
(12) Patent Application Publication
(10) Pub. No.: US 2025/0297205 A1
(43) Pub. Date: Sep. 25, 2025

(54) Apparatus and Method for Electroporation

(71) Applicant: TTP PLC, Melbourn (GB)

(72) Inventors: Debbie McArthur, Melbourn (GB); Verity Jackson, Melbourn (GB); Richard Janse Van Rensburg, Melbourn (GB)

(21) Appl. No.: 18/862,371

(22) PCT Filed: May 3, 2023
(86) PCT No.: PCT/GB2023/051174

§ 371 Date: Nov. 1, 2024

(30) Foreign Application Priority Data
May 3, 2022 (GB) 2206443.0

(51) Int. Cl. C12M 1/42

(52) U.S. Cl. CPC C12M 35/02

(57) ABSTRACT

An electroporation apparatus is disclosed, comprising:
an electroporation channel for carrying cells in a liquid, the electroporation channel comprising:
   a first inlet arranged to introduce the cells and a first supply of liquid into the electroporation channel,
   an outlet arranged to receive the cells from the electroporation channel, and
   a second inlet arranged to introduce a second supply of liquid into the electroporation channel, the second inlet being located between the first inlet and the outlet;
wherein the electroporation apparatus further comprises a first electrode which is configured to be electrically connected to the electroporation channel via the first inlet or the outlet and a second electrode which is configured to be electrically connected to the electroporation channel via the second inlet; and
wherein the first and second electrodes are configured to provide a potential difference between the second inlet and the first inlet, or between the second inlet and the outlet, such that, in use, an electric field is generated in, and substantially parallel to, the electroporation channel.

This invention relates to an apparatus and method. In particular, it relates to a cell electroporation apparatus and methods for using the same.

### BACKGROUND

### Cell Electroporation

Electroporation is a microbiology technique in which an electric field is applied to cells in order to increase the permeability of the cell membrane. This allows chemicals, for example, drugs, electrode arrays or nucleic acids (such as DNA), to be introduced into the cell (also called electrotransfer). This process is often used to transform bacterial, yeast or plant protoplasts by introducing new coding DNA. It is also widely used to transfect foreign DNA into eukaryotic cells, in particular mammalian cells.

To perform electroporation, cells are typically suspended in a conducting buffer between two electrodes and an external electric field is applied to induce a transmembrane potential. When this transmembrane potential exceeds a certain value, the membrane is permeabilised. Under appropriate electrical conditions, cell permeabilisation is reversible, ensuring survival of the cells.

### Cuvette Devices

Conventionally, electroporation is conducted in a cuvette-based apparatus. Cuvette electroporation systems typically produce distorted electric fields which induce local pH variation in the electroporation buffer and variable metal ion dissolution. Additionally, cuvette electroporation systems can suffer from cavitation and excess heat generation. Furthermore, cuvette electroporation systems are inappropriate for mass screening-type experiments.

### Flow-Based Devices

Flow-based electroporators typically comprise a chamber where an electric field is generated between two oppositely charged electrodes. The suspension of cells to be electroporated is flowed through the electric field, with the suspension in contact with the electrodes.

One problem with electroporation devices, including flow-based devices, is maintaining a consistent and accurately controllable electric field. Non-uniform electric fields can result in exposure of individual cells within a population to excessive electric field which will cause prolonged or irreversible electroporation, leading to cell death and/or phenotypic changes. Additionally, contact between cells and electrodes can result in similar undesirable effects.

### Capillary Devices

Capillary electroporation devices are also known but these are poorly suited to high throughput experimentation since cells can only be electroporated in small batches.

There therefore remains a need to provide a system that can perform electroporation with improved cell viability in electroporated cells.

### SUMMARY

The apparatus of the present invention addresses the above-mentioned problems. The apparatus is readily scalable, can operate continuously and improves cell viability.

In a first aspect the present invention provides an electroporation apparatus comprising: an electroporation channel for carrying cells in a liquid, the electroporation channel comprising: a first inlet arranged to introduce the cells and a first supply of liquid into the electroporation channel, an outlet arranged to receive the cells from the electroporation channel, and a second inlet arranged to introduce a second supply of liquid into the electroporation channel, the second inlet being located between the first inlet and the outlet. The electroporation apparatus further comprises a first electrode which is configured to be electrically connected to the electroporation channel via the first inlet or the outlet, and a second electrode which is configured to be electrically connected to the electroporation channel via the second inlet. The first and second electrodes are configured to provide a potential difference between the second inlet and the first inlet, or between the second inlet and the outlet, such that, in use, an electric field is generated in, and substantially parallel to, the electroporation channel.

The apparatus of the invention improves cell viability and is easily scalable.

In use, cells enter the electroporation channel via one or more of the inlets and exit via one or more of the outlets.

The lengths of the channels may be defined as follows. L1 is the distance from the first inlet to the second inlet and L2 is the distance from the second inlet to the outlet. L1 can be the same or different to L2. L1 can be greater than L2, or L2 can be greater than L1.

In a second aspect, the present invention provides an electroporation apparatus comprising an electroporation channel for carrying cells in a liquid, the electroporation channel comprising: a first inlet for introduction of the cells and a first supply of liquid into the electroporation channel, and an outlet for removing the cells and the liquid from the electroporation channel. The electroporation apparatus further comprises a first electrode which is configured to be electrically connected to the channel via the first inlet, a second electrode which is configured to be electrically connected to the electroporation channel between the first inlet and the outlet, and a third electrode which is configured to be electrically connected to the electroporation channel via the outlet. The first and second electrodes are configured to provide a potential difference (X1) between the first inlet and a position in the electroporation channel at a distance L1 from the first inlet, such that, in use, a first electric field (E1) is generated in and substantially parallel to the electroporation channel between the first inlet and the position at a distance L1 from the first inlet. The second and third electrodes are configured to provide a potential difference (X2) between the outlet and a position in the electroporation channel at a distance L2 from the outlet, such that, in use, a second electric field (E2) is generated in and substantially parallel to the electroporation channel between the outlet and the position at a distance L2 from the first outlet. L1≠L2 and/or X1≠X2.

Similar to the apparatus of the first aspect, the apparatus of the second aspect is able to effectively provide electroporation to cells whilst maintaining cell viability. The devices can also be easily scaled.

Also provided in accordance with the present invention is a cell electroporation method comprising:
a) providing an electroporation apparatus as described above;
b) supplying a substantially constant flow of cell-containing liquid to the first inlet;
c) applying a potential difference between at least two of the electrodes; and
d) collecting the cell-containing liquid at the outlet.

### FIGURES

FIG. 1—Shows an example bulk flow electroporation device of the prior art

FIGS. 2 to 6—Show various electroporation devices according to the present invention.

FIG. 7—Shows the output of a 3D finite element electric model.

FIG. 8—Shows the electric field strength and potential plotted along a sample line running along the centre of the electroporation channel for a device as shown in FIG. 3.

FIG. 9—Shows an embodiment of the invention wherein the apparatus comprises multiple electroporation devices.

FIG. 10—Shows example method steps.

FIG. 11—Shows the results of an example transfection.

FIG. 12—Shows an embodiment in which two electroporation devices have been linked in series.

FIGS. 13 to 15—Describe embodiments of the invention in detail.

### DETAILED DESCRIPTION

FIG. 1 shows an example flow-based electroporation device according to the prior art. The device 2 comprises a high voltage electrode 4 and a ground electrode 6. In use, cells which enter the channel at inlet I then pass electrode 6, they then travel the length of the channel L before passing high voltage electrode 4 and exiting the channel at outlet O. An electric field E (direction shown by arrow E) is generated in the channel 8.

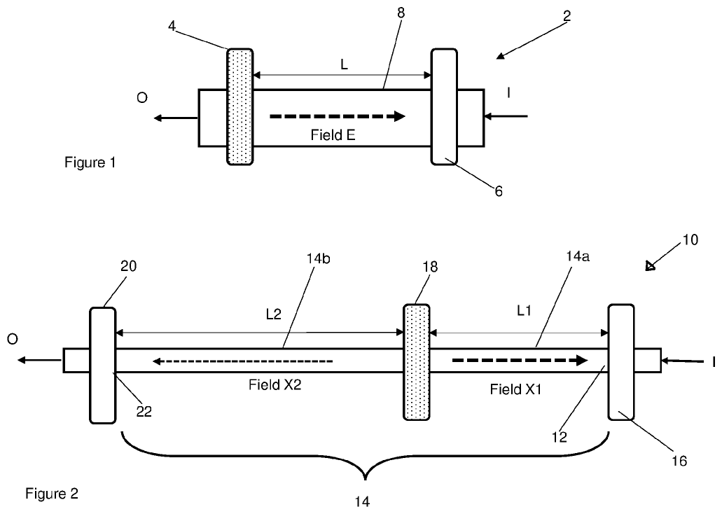

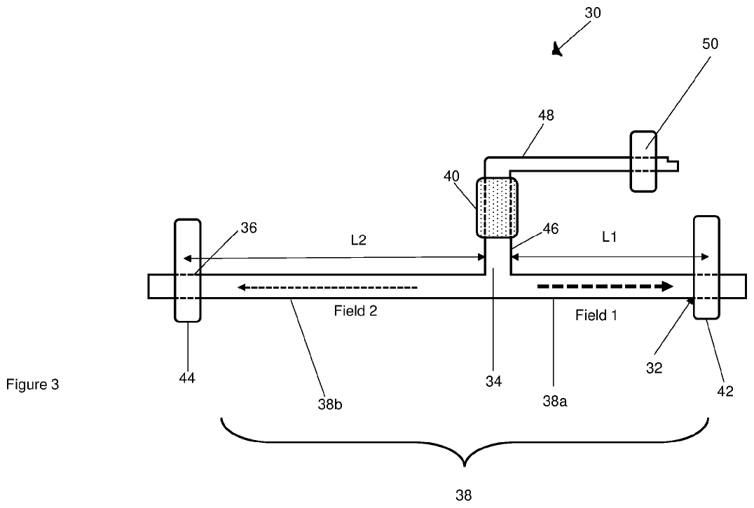

FIG. 2 shows an example electroporation apparatus 10. The apparatus comprises a first inlet 12 configured for introducing cells into an electroporation channel 14a,b. The apparatus also includes an outlet 22 configured to receive cells from the electroporation channel 14a,b, the outlet 22 being disposed at an opposite end of the electroporation channel 14 to the inlet. A first electrode 16 is electrically coupled to the first inlet 12. A second electrode 18 is positioned at a distance L1 from the inlet 12. The second electrode 18 is electrically connected to the electroporation channel 14 and defines a first section 14a of the electroporation channel which connects the inlet 12 to the second electrode 18 and a second section 14b of the electroporation channel which connects the second electrode 18 to the outlet 22. A third electrode 20 is situated at the outlet 22. In use, a potential difference is generated between the first and second electrodes to generate an electric field X1 in the first section 14a. A second electric field X2 is generated between the second and third electrodes.

In some embodiments the first 16 and third 20 electrodes are ground electrodes. This means that fluid will exit the system at ground potential. The strengths of the electroporation fields in the two channel sections 14a and 14b are independent of the solution conductivity because the system is now a voltage divider. The two different lengths enable certain electroporation protocols to be implemented e.g. ‘shock+coast’ procedure wherein the cells are first subjected to a high electric field and then to a lower electric field. This involves application of a higher electric field for a shorter time and a lower electric field for a longer time. This is useful because the cell pores are opened up by the higher voltage and then kept open by the extended application of lower voltage, to facilitate mass transfer of payload (e.g. of DNA) into the cells.

The electric field in the channel can be calculated using formula (I) below, where E is the electric field (V/m), V is the potential difference between the electrodes (V), and L is the length of the electroporation channel in which electroporation takes place. In some examples, the length of the electroporation channel is considered to be the length over which the electric field is substantially uniform (electroporation length). The size of electric field necessary to perform cell electroporation will depend on the cell type. For example, 25 to 150 kV/m, or between 30 kV/m-100 kV/m is appropriate for some mammalian cells.

$$E = V / L , where \\ E = field strength \\ V = voltage \\ L = electroporation length \qquad ( I )$$

The input rate of cells in the channel can be determined by calculating the time required for cells to be electroporated at a given field strength and using a given length of channel. The following formula (II) can be used:

$${Q}_{cells} = ( H . W . L ) / T , where \\ H = channel depth \\ W = channel width \\ L = electroporation length \\ T = electroporation time ( or residency time ) \qquad ( II )$$

For example:

| | | |
|---|---|---|
| H | 0.25 | mm |
| W | 0.5 | mm |
| L | 5.00 | mm |
| T | 50 | ms |
| Qcells | 750 | ul/min |

FIG. 3 shows an example electroporation apparatus 30. The apparatus 30 includes an electroporation channel 38 which comprises a first inlet 32, a second inlet 34, and an outlet 36. The apparatus 30 also comprises first, second and third electrodes 42, 40, 44 that are electrically connected to the electroporation channel 38 in order to generate an electric field in the electroporation channel 38. The first inlet 32 is electrically connected to the first electrode 42. The third electrode 44 is electrically connected to the outlet 36. In this example, the first and third electrodes 42, 44 are configured to make direct contact with any liquid in the electroporation channel 38 at the first inlet 32 and outlet 36, respectively, in order to make the electrical connection to the electroporation channel.

The second electrode 40 is electrically and fluidly connected to the second inlet 34 by way of a conduction channel 46.

In some examples, the first and third electrodes 42, 44 make direct contact with the first inlet 32 and outlet 36 respectively. In other examples, one or both of the first and third electrodes are electrically and fluidly connected to first inlet 32 and/or outlet 36 by way of a respective conduction channel.

The inlets and outlets can have any shape.

A buffer channel 48 (also referred to as a second channel) is fluidly connected to the second electrode 40 and a fourth electrode 50. In use the buffer channel carries fluid, into the electroporation channel. In some embodiments, the buffer channel does not carry cells. The fluid in the buffer channel 48 can provide thermal cooling to the second electrode.

The first 42, third 44 and fourth 50 electrodes, i.e., the electrodes at inlets and outlets to the system, are preferably ground electrodes. The second electrode 40 is preferably a high voltage electrode, for example the electrode may have a voltage between 100V to 500V relative to the ground electrode. Since the cells in the electroporation channel 38 do not contact the high voltage second electrode 40, electrochemical effects on the cells are minimised. The inflowing buffer provides a passive, chemically compatible, non-metallic current connection to the electroporation channel. The temperature of the buffer which is provided to buffer channel 48 through the buffer inlet BI can be used to mitigate the effects of ohmic heating in the electroporation channel 38.

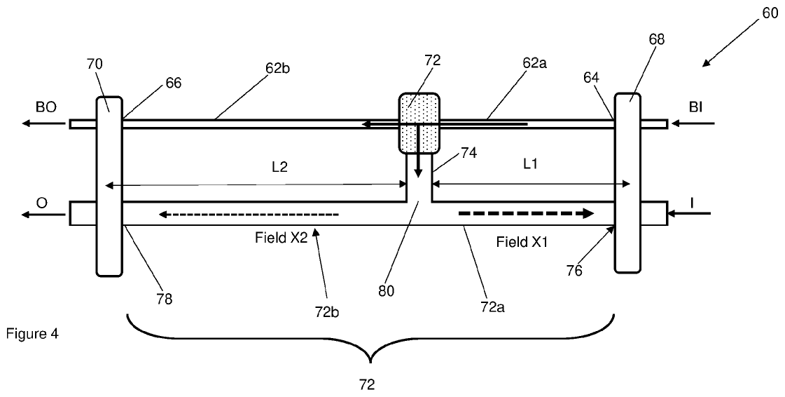

FIG. 4 illustrates a further development of the apparatus in FIG. 3. FIG. 4 shows an electroporation apparatus 60 which comprises a buffer channel 62a,b (also referred to as a second channel) with an inlet 64 and an outlet 66 which are electrically coupled to a first electrode 68 and a third electrode 70, respectively. A second electrode 72 is configured to be electrically connected to the electroporation channel at a length L1 from the first inlet 64 and at a length L2 from the outlet 66. The second electrode is in contact with the buffer channel 62a,b. An electroporation channel 72a,b is fluidly connected to the second electrode and the buffer channel 62a,b by way of a conduction channel 74. The conduction channel electrically couples the second electrode to the electroporation channel. The electroporation channel 72a,b has a first inlet 76, a second inlet 80 and an outlet 78. The first inlet 76 is in contact with to the first electrode 68. The second inlet 80 is electrically and fluidly connected to the second electrode 72. The outlet 78 is in contact with the third electrode 70.

Some of the advantages of the apparatus in FIG. 4 are that electrochemical side-reaction products can be directly flushed to waste or for re-cycling. Additionally, due to the separate buffer channel a high buffer throughflow can be achieved resulting in a higher degree of cooling to the high voltage second electrode 72. Since the buffer flow path and the electroporation channel containing the cells are essentially decoupled, different buffer solutions can be used in each channel.

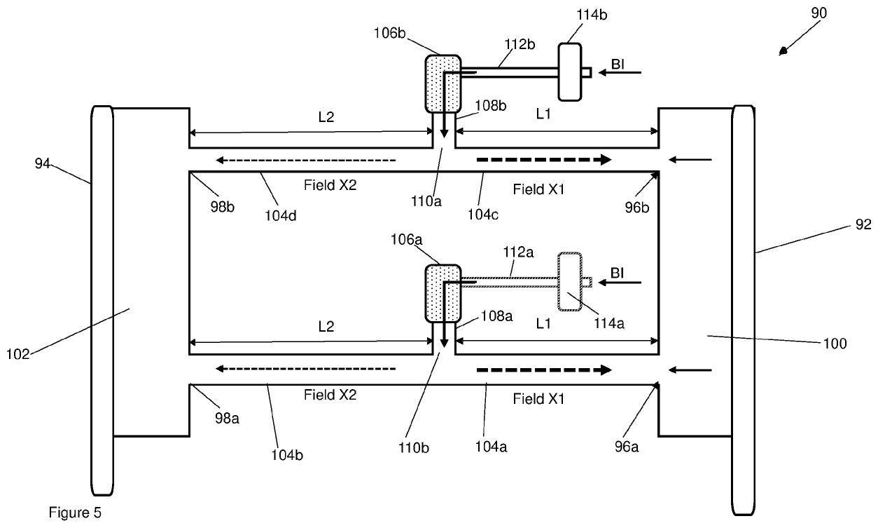

The device 90 shown in FIG. 5 is a further embodiment of the present invention. In this embodiment the first 92 and third 94 electrodes are both electrically connected to the first inlet 96a,b and the outlet 98a,b by way of buffer reservoirs 100 and 102. The buffer reservoirs have dimensions such that the voltage drops between the first electrode 92 and the inlet 96a,b and between the third electrode 94 and the outlet 98a,b are negligible. The potential difference is therefore effectively applied at the inlet 96a,b and outlet 98a,b. This separation of the electroporation channel 104a,b,c,d from the physical electrode means that the cells and electroporation buffer need not touch the electrodes, thereby reducing the amount of unwanted electrochemical reactions which occur.

Between the reservoirs 100 and 102 are two devices similar to those described with respect to FIG. 3. The said two devices comprise electroporation channels 104a,b,c,d and second electrodes 106a,b, which are electrically linked by a conduction channel 108a,b to a second inlet 110a,b. A buffer channel 112a,b links the second electrode 106a,b to a ground electrode 114a,b.

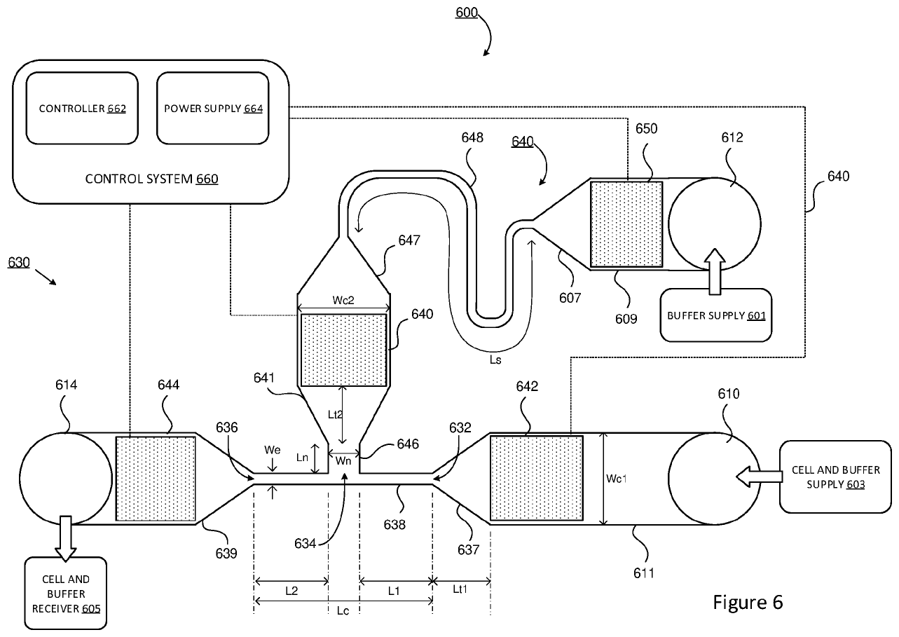

FIG. 6 illustrates an electroporation system 600, which includes an electroporation apparatus 630 and a control system 660 for controlling the operation of the electroporation apparatus 630. For ease of understanding, the control system 660 is illustrated schematically, and the electroporation apparatus 630 is shown using a simplified section view.

The electroporation apparatus 630 includes an electroporation channel 638 which comprises a first inlet 632, a second inlet 634 and an outlet 636. In an analogous way to the examples described above, the second inlet 634 is at a location of the electroporation channel between the first inlet 632 and the outlet 636. The distance between the first inlet 632 and second inlet 634 defines a first electroporation length L1, and the distance between the second inlet 634 and outlet 636 defines a second electroporation length L2.

The apparatus 630 includes a first port 610 and a grounded input channel 611 (also referred to as a first electrode channel 611). The first port 610 connects to the first inlet 632 via the grounded input channel 611, such that liquid (e.g. cell and buffer solution) can flow into the first inlet 632 via the first electrode channel 611. The grounded input channel 611 has a greater cross-sectional area than the electroporation channel 638. The grounded input channel 611 includes a grounded triangular section 637 (also referred to as a constriction section) which provides connection to the first inlet 632 of the electroporation channel 638. The grounded triangular section 637 provides a graduated change in cross-sectional area between the electroporation channel 638 and the grounded input channel 611.

A first electrode 642 is disposed in the grounded input channel 611 such that liquid present in the first electrode channel can contact the first electrode 642. In this example, the first electrode 642 provides a ground potential to the grounded input channel 611.

The system 600 further includes a cell and buffer supply 603 which supplies cells suspended in a buffer to the apparatus 630. The cell and buffer supply 603 is fluidly connected to the first port 610 in order to supply cells in buffer to the electroporation channel 638 via the grounded input channel 611.

The apparatus 630 further includes a high voltage input channel 615 (also referred to as a second electrode channel) which is connected to the second inlet 634, such that liquid (e.g. buffer solution) can flow into the second inlet 634 via the high voltage input channel 615. The high voltage input channel 615 has a greater cross-sectional area than the electroporation channel 638. The high voltage input channel 615 includes a neck triangular section 641 which provides connection to the second inlet 634 of the electroporation channel 638, via a neck 646. The neck triangular section 641 provides a graduated change in cross-sectional area between the high voltage input channel 615 and the neck 646.

In this example, the neck 646 has a cross sectional area which is greater than the electroporation channel 638. This is advantageous because the larger area of the neck ensures that there is a reduced voltage drop between the high voltage electrode and the electroporation channels.

A second electrode 640 is disposed in the high voltage input channel 615 such that liquid present in the high voltage input channel 615 can contact the second electrode 640. In this example, the second electrode 640 provides a high voltage (or a voltage which is higher than ground) to the high voltage input channel 615.

Exemplary dimensions of the apparatus 630 are provided in the table below.

The high voltage input channel 615 is supplied with liquid, such as buffer, from a second port 612 which is connected to a buffer supply 601. The second port 612 is connected to the high voltage input channel 615 via a grounded buffer channel 609 (also referred to as a fourth electrode channel) and a high resistance section 640.

A fourth electrode 650 is disposed in the grounded buffer channel 609 such that liquid present in the grounded buffer channel 609 can contact the fourth electrode 650. In this example, the fourth electrode 650 provides a ground potential to the grounded buffer channel 609.

The high resistance section 640 has a narrow, serpentine configuration in order to provide high resistance to current. This protects the buffer supply 601 and other components from high voltage.

The buffer supply 601 supplies buffer to the apparatus 630. The buffer may be the same or different buffer to that of the cell and buffer supply 603.

The apparatus 630 also includes a third port 614 and a grounded output channel 613 (also referred to as a third electrode channel). The third port 614 connects to the outlet 636 via the grounded output channel 613, such that liquid (e.g. cell and buffer solution) can flow from the outlet 636 to the third port 614 via the grounded output channel 613. The grounded output channel 613 has a greater cross-sectional area than the electroporation channel 638. The grounded output channel 613 includes a grounded triangular section 639 (also referred to as a constriction section) which provides connection to the outlet 636 of the electroporation channel 638. The grounded triangular section 639 provides a graduated change in cross-sectional area between the electroporation channel 638 and the grounded output channel 613.

A third electrode 644 is disposed in the grounded output channel 613 such that liquid present in the grounded output channel can contact the third electrode 644. In this example, the third electrode 644 provides a ground potential to the grounded output channel 613.

The voltage-control section is configured to apply a voltage V to the apparatus 630 which is higher than a ground voltage (example 100V to 500V higher than the ground voltage) while managing any undesirable effects caused by the high voltage.

Electric field strength is inversely proportional to the cross-sectional area of the buffer-filled channel. Therefore, the electric field E is higher in the reduced cross-sectional area of the electroporation channel 638 compared to other sections of the system (such as the grounded input channel 611).

The width We of the electroporation channel 638 may be, for example, 0.5 mm and the depth of the channel 638 may be 0.25 mm.

The channels 611, 609, 615, 613 has a width Wc1 which is wider than the width We of the electroporation channel 638, to allow greater contact between the electrodes and liquid in the channel and reduced electric field strength. As an example, the grounded input channel 611 may have a width Wc1 of 5 mm and may have a depth of 0.25 mm.

The electroporation system 600 includes the control system 660 for controlling the operation of the electroporation apparatus 630. The control system 660 comprises a controller 662 and a power supply 664, any may include further elements which have been omitted for simplicity. The control system 660 is connected to each of the four electrodes of the electroporation apparatus 630 via connections shown with dotted lines. The controller 662 controls the electrical signals supplied to the electrodes, and may also control the supply of buffer and cells into the respective ports. In this way, the control system 660 allows cells to undergo electroporation in a configurable manner.

| Label in | | | | | |
|---|---|---|---|---|---|
| FIG. 6 | Name | Example Value | | Example Range | |
| | Channel depth (of all | 0.25 | mm | 0.25-1.00 | mm |
| | channels) | | | | |
| We | Electroporation Channel | 0.5 | mm | 0.25-1.00 | mm |
| | width | | | | |
| Wc2 | High voltage input channel | 5 | mm | 1-10 | mm |
| | width | | | | |
| Wc1 | Grounded channel width | 5 | mm | 1-10 | mm |
| Wn | Neck width | 0.5 | mm | 0.5-5 | mm |
| Ln | Neck length | 1.75 | mm | 0.5-2 | mm |
| Lt2 | Neck Triangular length | 3 | mm | 2-5 | mm |
| Lt1 | Grounded Triangular length | 3 | mm | 2-10 | mm |
| Ls | Serpentine length | 24.49 | mm | 20-50 | mm |
| Lc | Channel length | 10 | mm | 6.5-25 | mm |
| | Channel Area | 0.125 | mm\hat{ }2 | | mm\hat{ }2 |
| L1 | First Electroporation length | 4.75 | mm | 3-10 | mm |
| L2 | Second Electroporation | 4.75 | mm | 3-10 | mm |
| | length | | | | |
| | Cell inflow from cell and | 750 | ml/min | 500-1500 | ml/min |
| | buffer supply | | | | |
| | Second inflow from buffer | 375 | ml/min | 100-750 | ml/min |
| | supply | | | | |
| | L1 average speed | 0.1 | m/s | 0.06-0.2 | m/s |
| | L2 average speed | 0.15 | m/s | 0.06-2.6 | m/s |
| | L1 Cell Residence time | 47.5 | ms | 20-65 | ms |
| | L2 Cell Residence time | 31.66667 | ms | 15-55 | ms |
| | Conductivity | 0.0271 | S/m | 0.025-0.1 | S/m |
| | Vᵢₙₚᵤₜ | 350 | V | 250-400 | V |
| | Rᵢₙₚᵤₜ | 1320.433 | kOhms | 2761100 | kOhms |
| | E Field | 31.3 | kV/m | 30-50 | kV/m |
| | Current | 0.27 | mA | 0.36-1.5 | mA |

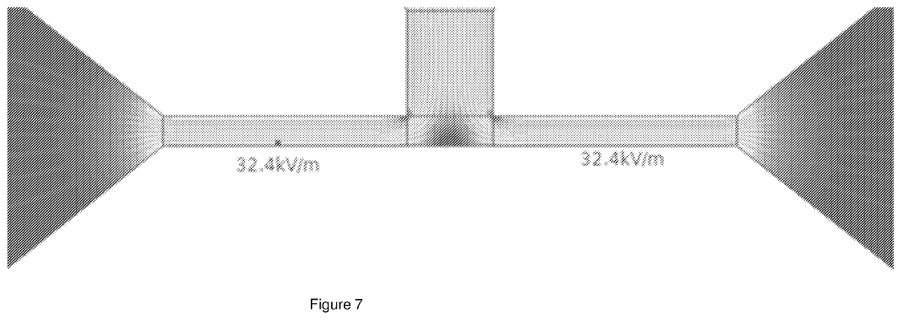

FIG. 7 shows the output of a 3D finite element electric model of the system. An example electric field strength in the electroporation channels is given as 32.4 kV/m on the figure, and a set of constant potential lines are also plotted. The electric field in exemplary apparatuses of the invention was modelled using the following parameters and the following software: COMSOL V5.6 Commercially available finite element package, Microsoft EXCEL spreadsheet, and MATLAB R2020a.

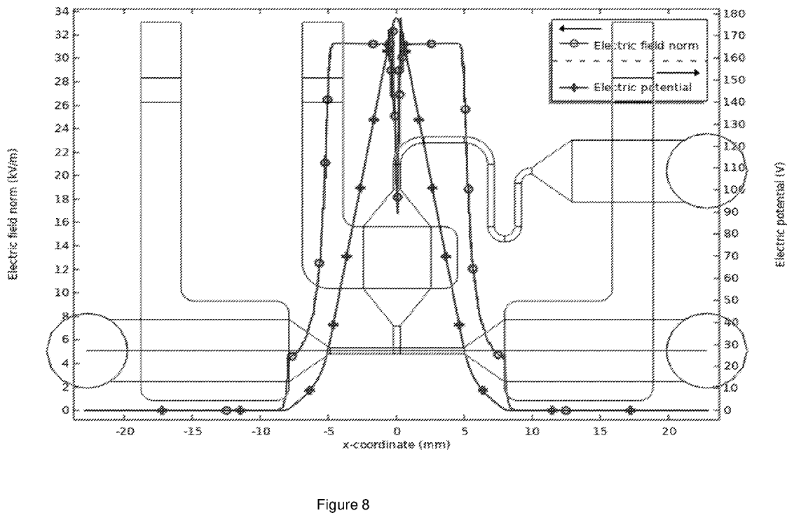

FIG. 8 shows the electric field strength and potential plotted along a sample line running along the centre of the electroporation channel for a device as shown in FIG. 3. The graphs are superimposed on top of the system in plain view to illustrate how these fields change along the channel. It can be seen that the electric field is substantially constant along the two electroporation channels L1 and L2.

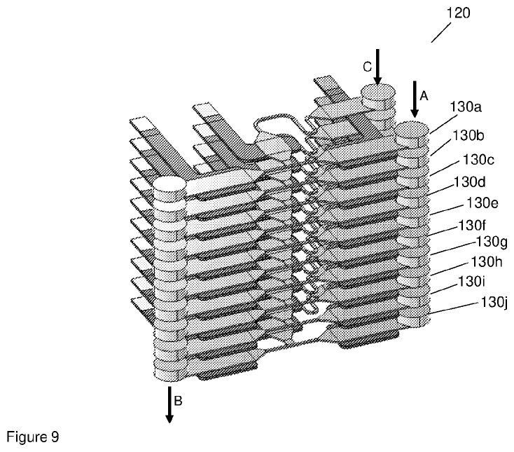

FIG. 9 shows an example electroporation apparatus 120 comprising multiple individual electroporation devices 130a-j. The devices can be set up in parallel. Arrow A indicates the inflow of the cell and liquid, arrow B indicates where the cells and liquid leave the apparatus. Arrow C shows a buffer-only inlet which is fluidly connected to buffer-only channels on each individual device 130a-j.

FIG. 9 shows example method steps for using the apparatus of the invention.

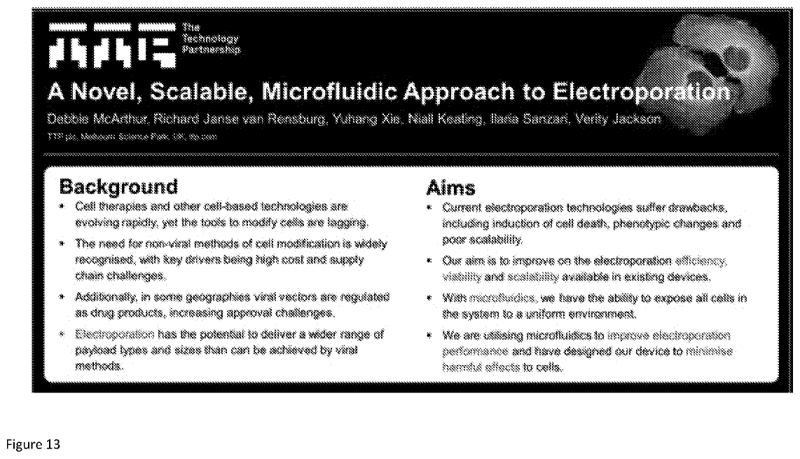

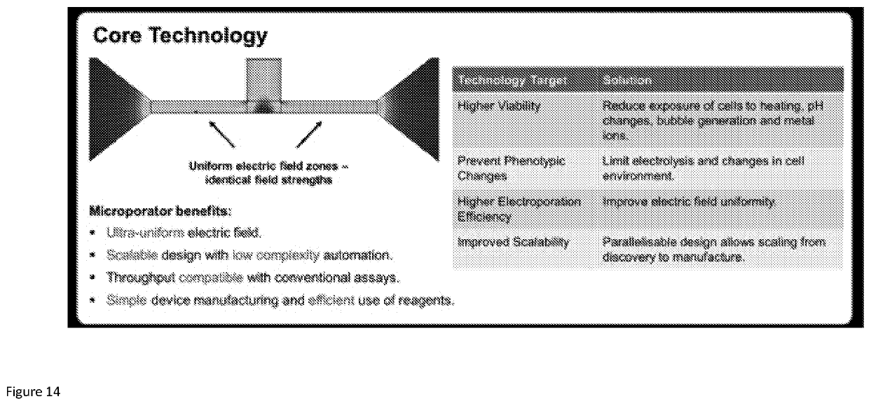

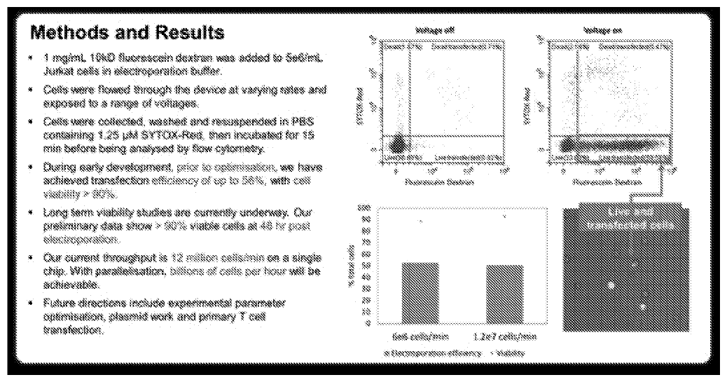

FIGS. 13 to 15 show a summary of certain embodiments of the invention. The text of FIGS. 13 to 15 is as follows:

### Background

Cell therapies and other cell-based technologies are evolving rapidly, yet the tools to modify cells are lagging.
The need for non-viral methods of cell modification is widely recognised, with key drivers being high cost and supply chain challenges.
Additionally, in some geographies viral vectors are regulate as drug products, increasing approval challenges.
Electroporation has the potential to deliver a wider range of payload types and sizes than can be achieved by viral methods.

### Aims

Current electroporation technologies suffer drawbacks, including induction of cell death, phenotypic changes and poor scalability.
Our aim is to improve on the electroporation efficiency, viability and scalability available in existing devices.
With microfluidics, we have the ability to expose all cells in the system to a uniform environment.
We are utilising microfluidics to improve electroporation performance and have designed our device to minimise harmful effects to cells.

### Core Technology

### Microporator Benefits:

Ultra-uniform electric field.
Scalable design with low complexity automation.
Throughput compatible with conventional assays.
Simple device manufacturing and efficient use of reagents.

| Technology Target | Solution |
|---|---|
| Higher Viability | Reduce exposure of cells to heating, pH |
| | changes, bubble generation and metal ions. |
| Prevent Phenotypic | Limit electrolysis and changes in cell |
| Changes | environment |
| Higher Electroporation | Improve electric field uniformity |
| Efficiency | |
| Improved Scalability | Parallelisable design allows scaling from |
| | discovery to manufacture |

### Methods and Results

1 mg/mL 10 kD fluorescein dextran was added to 5e6/mL Jurkat cells in electroporation buffer.
Cells were flowed through the device at varying rates and exposed to a range of voltages.
Cells were collected, washed and resuspended in PBS containing 1.25 μM SYTOX-Red, then incubated for 15 min before being analysed by flow cytometry.
During early development, prior to optimisation, we have achieved transfection efficiency of up to 58%, with cell viability >90%.
Long term viability studies are currently underway. Our preliminary data shows >90% viable cell at 48 hr post electroporation.
Our current throughput is 12 million cells/min on a single chip. With parallelisation, billions of cells per hour will be achievable.
Future directions include experimental parameter optimisations, plasmid work and primary T cell transfection.

### EXAMPLES

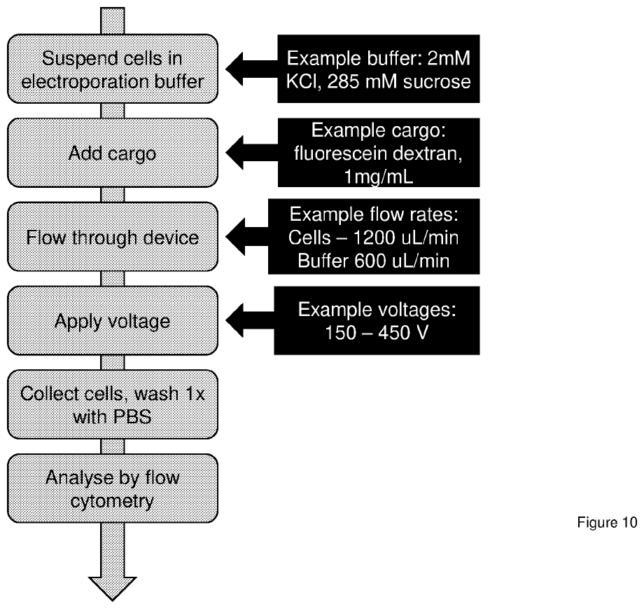

Cells were placed into an apparatus according to FIG. 6a and a protocol as described in FIG. 10 was performed.

1 mg/mL fluorescein dextran obtained from ThermoFisher was added to 5e6/mL Jurkat cells in electroporation buffer. The cells were obtained from Sigma-Aldrich (Merck). Cells were flowed through the device at a speed of 1200 μL/min and exposed to an electrical pulse of varying voltages between 200 V and 400 V.

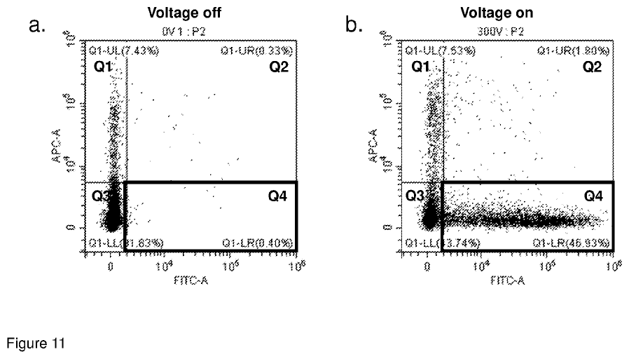

Following electroporation, the cells were analysed by flow cytometry to determine electroporation efficiency. Cell viability is assessed by SYTOX red staining. The results are shown in FIG. 11 Panel a) demonstrates the results when no voltage was applied (Voltage off (0V)) and panel b) demonstrates the results when a voltage of 300V was applied (Voltage on (300V)).

Cells in Q1 are dead and untransfected, cells showing in Q2 are dead and transfected, cells in Q3 are live but untransfected, and cells in Q4 are live and transfected. As can be seen after the application of 300V, more cells show in Q4, i.e., cells are live and transfected.

The representative plots in FIG. 10 show a transfection efficiency of 46.93% following exposure to 300V.

### Modifications and Alternatives

The following modifications and alternatives may be made to the aspects of the invention described above. These modifications are not intended to be limiting and can be used in combination or individually.

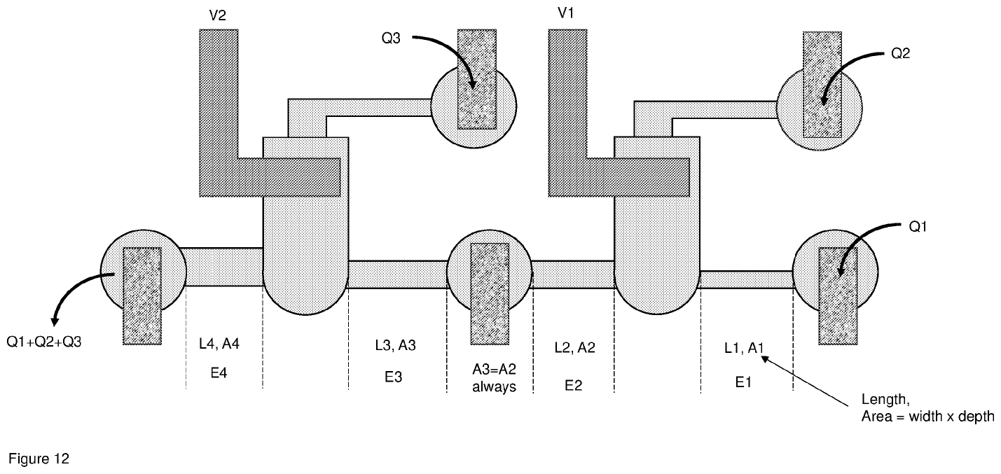

The cross-sectional area of the electroporation channels may be the same or the cross-section area may change—for example in the second length L2 the cross sectional area may be greater than in the first length L1. This accommodates the addition of extra buffer at the second inlet, and can be chosen to keep the speed of cell flow the same in both channels, i.e., the residence time. FIG. 12 is an example in which two electroporation devices have been linked in series. Because of the inflow from the electrode channels Q2 and Q3, the downstream electroporation channels (E2, E3 and E4) increase in width so that the residence times remain the same in all channels.

In general, the electroporation channel can be designed to allow varied speeds of buffer flow and varied cell residence times in L1 and L2. In one example, the width We of the electroporation channel is 0.5 mm in L1 and 0.75 mm in L2.

In some embodiments, the electrodes are attached to the inlets and/or outlet. In other embodiments, the electrode may be electrically coupled to the inlets and/or outlet when a conducting medium is present. It is not necessary for an electrode to be physically connected to the inlets and/or outlet. If the electrodes are electrically coupled to the inlets and/or outlet, the cells do not come into direct contact with the electrodes. This limits electrochemical processes in the cells, thereby improving the viability of the cells.

The electrodes may be electrically coupled to the inlets and/or outlet by way of a conduction channel (also referred to as inlet/outlet channels). The conduction channel may have a geometry such that there is negligible voltage drop between the electrode and the inlets and/or outlet.

In some embodiments of the electroporation apparatus according to the first aspect, the first electrode is electrically connected to the electroporation channel via the first inlet. The apparatus may further comprise a third electrode which is electrically connected to the electroporation channel via the outlet. The second and third electrodes are configured to provide a potential difference between the second inlet and the outlet, and the first and second electrodes are configured to provide a potential difference between the first inlet and the second inlet.

The inclusion of a third electrode enables the generation of two directionally- and potential-independent fields within the electroporation channel defined between the first inlet and the second inlet, and that defined between the second inlet and the outlet. The different electric fields can be independently controlled according to the electroporation protocol required. For example, the cells may be subjected to a “shock and coast” protocol, wherein the cells are first subjected to a high electric field (for example, in the part of the channel of length L1) and then subjected to a weaker electric field (for example, in the part of the channel of length L2).

In some embodiments, the conduction channel is filled with any conducting material. For example, an ionic buffer, conducting gels, a Pluronic (such as Pluronic F127), or hydrogels (including functionalised derivatives such as polyacrylamide, agarose, alginate, or chitosan).

The temperature of the liquid entering the conduction channel may be controlled, for example by providing a heating or cooling device. In this way it is possible to control the temperature in the electroporation channel and thereby reduce the effects of ohmic heating on the cells.

In some embodiments, the second electrode is connected to the second inlet. In such embodiments, the second inlet may be configured to supply buffer (but not cells) to the electroporation channel. By not introducing cells through the second inlet there is no direct contact between the cells and the second electrode. This stops the cells undergoing electrochemical processes and therefore improves cell viability. The dimensions of the second inlet may be chosen to minimise any current loss between the electrode and the main body of the electroporation channel. The cross-sectional area of the opening or junction between the second inlet and the electroporation channel should be large enough to minimise voltage loss (electrical resistance) between the second inlet and the electroporation channel. The length of any conduction channel between the second electrode and the second inlet should be short enough to minimise ohmic heating.

In some embodiments of the electroporation apparatus of the second aspect, the apparatus further comprises a second inlet arranged to introduce a second supply of liquid into the electroporation channel, the second inlet being located between the first inlet and the outlet at a distance L1 from the first inlet. The second electrode is electrically connected to the electroporation channel via the second inlet.

The following embodiments can be used in combination with any of the first or second aspects of the invention described above.

The electroporation channel may have any length, width and height. The channel may have any cross sectional shape. It is preferred that the cross-sectional shape of the electroporation channel is constant across the length of the electroporation channel. By maintaining a constant electric field across the length of the channel, the cross-sectional area of the channel can be increased without altering the electroporation parameters. This means that the apparatus of the present invention is easily scalable.

The dimensions of the electroporation channel are not particularly limited. Preferably, the channel has a width of between 0.25 mm and 1 mm. For example, the channel has a height of between 0.25 mm and 1 mm. The complete length of the electroporation channel is preferably between 5 mm and 30 mm. L1 is preferably between 5 mm and 10 mm and L2 is preferably 5 mm and 20 mm.

The cross-sectional shape of the electroporation channel is not particularly limited. For example, the cross sectional shape can be square, rectangular, circular, triangular, or any other suitable shape.

In some embodiments, the second electrode is physically connected to the second inlet. In such embodiments, the second inlet may be configured to supply buffer (but not cells) to the channel. By introducing buffer only through the second inlet, cells do not have direct contact with the second electrode. This thereby limits undesirable electrochemical processes on or in the cells and improves cell viability. The dimensions of the second inlet may be chosen to minimise any current loss between the high-voltage electrode and the main body of the channel. Any connecting channel between the electrode and the channel junction must be wide enough to minimise voltage loss (electrical resistance) and its length must be short enough to minimise ohmic heating.

When an electrode is electrically coupled to any inlet or any outlet, the electrode may physically touch the inlet and/or outlet. Alternatively, the electrode may be electrically coupled to the inlet or the outlet by way of a conducting channel filled with a conducting material (for example, an electroporation buffer). Such use of a conducting channel minimises contact between cells and the electrode electrically coupled to the respective inlet/outlet.

The conducting channel geometry and material is configured so that there is no significant voltage drop between the electrode and the inlet into the electroporation channel.

In some embodiments, the first and third electrodes described above are ground electrodes. This ensures that any liquid and cells entering and leaving the electroporation channel are at ground potential.

In embodiments wherein the apparatus comprises a second inlet located between the first inlet and the outlet, the apparatus may further comprise a second outlet fluidly connected by a second channel to the second inlet. In use, the second channel will preferably not contain cells. The provision of a second channel enables a higher degree of cooling due to the ability to achieve higher buffer throughflow without affecting the electroporation of the cells. The provision of a second channel which is in physical contact with one or more electrodes also enables any electrochemical waste products to be directly flushed out of the system for disposal or recycling.

In some embodiments wherein the apparatus comprises a second inlet located between the first inlet and the outlet, the apparatus further comprises a third inlet electrically connected to the third electrode and located between the second inlet and the outlet. This third inlet generally does not transport cells and is useful in the case where the cells may be sensitive to the presence of an electrode, providing the capability to distance the third electrode from the electroporation channel, while maintaining an electrical connection to the electroporation channel, in a similar manner as the second inlet and electrode.

In some embodiments, at least one of the electrodes is a planar electrode. Preferably more than one, or all of the electrodes are planar electrodes. Planar electrodes have a reduced current density on the electrode which minimises the likelihood of electrode erosion.

The electrodes are preferably printed onto the surface of the apparatus. Suitable electrode materials include platinum, gold, aluminium, titanium, chromium, indium tin oxide (ITO) and mixtures thereof. Electrodes may be coated with any suitable electrode coating material. Examples of suitable electrode coating materials include SiO₂, Si₃N₄, and parylene.

In some embodiments, the apparatus further comprises at least one additional electrode located between the first inlet and the outlet, the at least one additional electrode being configured to be electrically coupled to the electroporation channel, and wherein the at least one additional electrode is configured to provide a potential difference with respect to a voltage at the first inlet such that, in use, at least one additional electric field is generated in and substantially parallel to the channel. The generation of additional electric fields enables a further variety of electroporation protocols to be performed.

Also forming part of the invention is an electroporation apparatus comprising at least two of the apparatuses described in connection with the first and/or second aspects above, wherein a single fluid reservoir is fluidly connected to the at least two first inlets. Coupling of multiple electroporation devices enables easy scale-up of the apparatus. It is envisaged that each individual electroporation apparatus could be surrounded by coolant. In some embodiments at least 3, at least 5, at least 10, or at least 15 electroporation devices comprise the electroporation apparatus.

In use, the electroporation process can be modulated by tuning the pulse amplitude, duration, frequency and shape of the electric field.

The electrodes may be made from any suitable metal or conducting non-metal such as a conducting gel.

The apparatus is preferably formed on or within a chip. That is, the channels, electrodes, inlets and outlets are all manufactured as a unitary device that can be connected to suitable liquid and cell supplies.

The chip can be made by any method known in the art. The dimensions of the finished chip depend on the dimensions of the channels.

The electroporation apparatus may be made out of any suitable material. Examples of such materials include elastomers, silicone, glass, thermoplastics, and resins. Suitable thermoplastics include cyclic olefin copolymer (COC), polycarbonate (PC), polystyrene (PS), polypropylene (PP), polyethylene terephthalate (PET), and high density polyethylene (HDPE). Examples of suitable resins include ostemers.

A cell electroporation method also forms part of the invention. The method may comprise:
e) providing an apparatus as described herein;
f) supplying a substantially constant flow of cell-containing liquid to the first inlet;
g) applying a potential difference between at least two of the electrodes; and
h) collecting the cell-containing liquid at the outlet.

In some embodiments, the cell-containing liquid is supplied with a flow rate of between about 1000 to about 5000 μL per minute.

In some embodiments, the potential difference applied is between about 150 V and about 450 V.

In some embodiments, the cell-containing liquid flow rate and a length of the electroporation channel may be arranged such that a residence time for the cell-containing liquid in the electroporation channel is between about 4 ms and about 100 ms.

The cell-containing buffer may be any buffer suitable for cell electroporation. Suitable buffers are commercially available or may be prepared. Electroporation may have formulations that mimic cellular cytoplasm composition; thus, enhancing pore resealing after electroporation and increase cell viability.

1. An electroporation apparatus comprising:
an electroporation channel for carrying cells in a liquid, the electroporation channel comprising:
a first inlet arranged to introduce the cells and a first supply of liquid into the electroporation channel,
an outlet arranged to receive the cells from the electroporation channel, and
a second inlet arranged to introduce a second supply of liquid into the electroporation channel, the second inlet being located between the first inlet and the outlet;
wherein the electroporation apparatus further comprises a first electrode which is configured to be electrically connected to the electroporation channel via the first inlet or the outlet and a second electrode which is configured to be electrically connected to the electroporation channel via the second inlet; and
wherein the first and second electrodes are configured to provide a potential difference between the second inlet and the first inlet, or between the second inlet and the outlet, such that, in use, an electric field is generated in, and substantially parallel to, the electroporation channel.

2. The electroporation apparatus of claim 1, wherein
the second electrode is configured to be electrically connected to the electroporation channel via an inlet channel which is connected to the second inlet.

3. The electroporation apparatus of claim 1, wherein the first electrode is electrically connected to the electroporation channel via the first inlet, and wherein the apparatus further comprises a third electrode which is electrically connected to the electroporation channel via the outlet, wherein the second and third electrodes are configured to provide a potential difference between the second inlet and at the outlet and the first and second electrodes are configured to provide a potential difference between the first inlet and the second inlet.

4. An electroporation apparatus comprising:
an electroporation channel for carrying cells in a liquid, the electroporation channel comprising:
a first inlet for introduction of the cells and a first supply of liquid into the electroporation channel, and
an outlet for removing the cells from the electroporation channel;
wherein the electroporation apparatus further comprises a first electrode which is configured to be electrically connected to the electroporation channel via the first inlet, a second electrode which is configured to be electrically connected to the electroporation channel at a location between the first inlet and the outlet, and a third electrode which is configured to be electrically connected to the electroporation channel via the outlet;
wherein the first and second electrodes are configured to provide a potential difference (X1) between the first inlet and a position in the electroporation channel at a distance L1 from the first inlet, such that, in use, a first electric field (E1) is generated in and substantially parallel to the electroporation channel between the first inlet and the position at a distance L1 from the first inlet;
wherein the second and third electrodes are configured to provide a potential difference (X2) between the outlet and the position in the electroporation channel at a distance L2 from the outlet, such that, in use, a second electric field (E2) is generated in, and substantially parallel to, the electroporation channel between the outlet and the position at a distance L1 from the first inlet; and
wherein L1≠L2 and/or X1≠X2.

5. The electroporation apparatus of claim 4, further comprising a second inlet arranged to introduce a second supply of liquid into the electroporation channel, the second inlet being located between the first inlet and the outlet at the distance L1 from the first inlet and a distance L2 from the outlet, wherein the second electrode is electrically connected to the electroporation channel via the second inlet.

6. The electroporation apparatus of claim 1, wherein the first electrode is a ground electrode.

7. The electroporation apparatus of claim 1, further comprising a second outlet fluidly connected by a second channel to the second inlet.

8. The electroporation apparatus of claim 3, wherein the apparatus comprises a second inlet located between the first inlet and the outlet and further comprises a third inlet electrically connected to the third electrode and located between the second inlet and the outlet.

9. The electroporation apparatus of claim 1, wherein at least one of the electrodes is a planar electrode.

10. The electroporation apparatus of claim 1, wherein the apparatus further comprises at least one additional electrode located between the first inlet and the outlet, the at least one additional electrode being electrically coupled to the electroporation channel, and wherein the at least one additional electrode is configured to provide a potential difference with respect to a voltage at the first inlet such that, in use, at least one additional electric field is generated in and substantially parallel to the electroporation channel.

11. A cell electroporation method comprising:
a) providing an apparatus of claim 1;
b) supplying a substantially constant flow of a cell-containing liquid to the first inlet;
c) applying a potential difference between at least two electrodes; and
d) collecting the cell-containing liquid at the outlet.

12. The cell electroporation method of claim 12, wherein the cell-containing liquid is supplied with a flow rate of between about 1000 to about 5000 μL per minute.

13. The cell electroporation method of claim 12, wherein the cell-containing liquid is supplied with a flow rate of between about 1000 to about 5000 μL per minute.

14. The cell electroporation method of claim 12, wherein the potential difference applied is between about 150 V and about 450 V.

15. The cell electroporation method of claim 12, wherein the cell-containing liquid flow rate and a length of the electroporation channel is arranged such that a residence time for the cell-containing liquid in the electroporation channel is between about 4 ms and about 100 ms.

16. The electroporation apparatus of claim 3, wherein the third electrode is a ground electrode.

17. An electroporation apparatus comprising at least two apparatuses of claim 4, wherein a single fluid reservoir is fluidly connected to the first inlet of each of the two apparatuses.

18. A cell electroporation method comprising:
a) providing an apparatus of claim 4;
b) supplying a substantially constant flow of a cell-containing liquid to the first inlet;
c) applying a potential difference between at least two electrodes; and
d) collecting the cell-containing liquid at the outlet.

19. The cell electroporation method of claim 18, wherein the cell-containing liquid is supplied with a flow rate of between about 1000 to about 5000 μL per minute.

20. The cell electroporation method of claim 18, wherein the potential difference applied is between about 150 V and about 450 V.

21. The cell electroporation method of claim 18, wherein the cell-containing liquid flow rate and a length of the electroporation channel is arranged such that a residence time for the cell-containing liquid in the electroporation channel is between about 4 ms and about 100 ms.
